(19) United States
(12) Patent Application Publication
(10) Pub. No.: US 2018/0104173 A1
(43) Pub. Date: Apr. 19, 2018

(54) A Polymer and a Composition Comprising the Polymer

(71) Applicant: BASF SE, Ludwigshafen am Rhein (DE)

(72) Inventors: Daniel Stadler, Mannheim (DE); Parag Kulkarni, Shanghai (CN); Peter Hoessel, Schifferstadt (DE); Mathias Gerst, Maikammer (DE)

(21) Appl. No.: 15/573,535

(22) PCT Filed: May 6, 2016
(86) PCT No.: PCT/EP2016/060169

§ 371 Date: Nov. 13, 2017

(30) Foreign Application Priority Data
May 18, 2015 (CN) PCT/CN2015/079203
Aug. 4, 2015 (CN) PCT/CN2015/086036
Aug. 21, 2015 (EP) 15181921.6

(51) Int. Cl. A61K 8/81; A61K 8/36; A61Q 5/06; A61K 8/39; C08F 220/18; C09J 133/06

(52) U.S. Cl. CPC A61K 8/8152; A61K 8/36; A61Q 5/06; C08F 218/08; C08F 220/18; C09J 133/06; C08F 2220/1858; A61K 8/39

(57) ABSTRACT

The present invention relates to a hair styling and restyling composition comprising a cosmetically acceptable pressure sensitive adhesive polymer with restyling performance and improved washout from hair, to a pressure sensitive adhesive polymer with glass transition temperatures of lower than −20° C., to a process for preparing said pressure sensitive adhesive polymer and to use of said pressure sensitive adhesive polymer for improving the restylability and washout behavior of a hair styling and restyling composition.

### FIELD OF THE INVENTION

The present invention relates to a polymer with restyling performance and improved washout from hair, to a hair styling and restyling composition comprising the polymer, to a process for preparing said polymer and to the use of said polymer for improving the restylability and washout behavior of a hair styling and restyling composition.

### BACKGROUND OF THE INVENTION

Hair styling includes forming hairstyles and keeping the formed hairstyles. Hair styling polymers with high glass transition temperatures (usually >60° C., often >100° C.) have been developed for forming strong and brittle films on hair and achieving a strong, long lasting hold. While hair styling polymers currently on the market are generally effective in holding the hair in a desired shape or configuration, many are not designed to allow the hairstyle to be later modified to a desired shape or reshaped into the original style without application of additional material or use of water. The hair styling polymers will lose styling functions once the brittle films are broken by applying a quite strong force. For this reason, brittle film-forming polymers are not suitable for restyling hair. Polymers that exhibit such a performance profile would have to be soft film forming and somehow tacky as well, which can be achieved by polymers with low glass transition temperature, some of which are so called pressure sensitive adhesive polymers.

DE 10 2006 058 389 A1 describes cosmetic compositions with vinylester/ethylene copolymers, modified polyacrylic esters and polymethacrylic esters as suitable styling agents to achieve restyleability.

US 2007/0224140 A1 describes the use of cosmetic compositions with adhesive microspheres for hair and eyelash styling with restyleability (reshapeability).

WO 2007/071308 A1 discloses a hair treatment composition comprising an acrylic pressure sensitive adhesive containing methylacrylate.

WO 2012/107366 A1 describes hair care compositions comprising: i) a polyacrylate cross polymer; and ii) an acrylic pressure sensitive adhesive comprising: a) an acrylic group having a side-chain with at least 4 carbons and; b) a short side-chain acrylic such as methyl acrylate, which impart restyling benefits to the hair.

On the other hand, wax formulations also allow styling and/or restyling and form very soft films that are constantly tacky and only glue together hair fibers.

DE 10 2008 016 065 A1 describes a product for temporary deformation and stabilization of the hairstyle in the form of a wax or a wax emulsion containing waxes, oils, greases and possibly emulsifiers.

EP 2 611 412 is directed to an acrylic copolymer which is suitable both as hair fixative/film former and a thickening agent in hair styling and personal care compositions.

Furthermore, when a hair styling and/or restyling composition needs to be removed from the hair, it should be easily washed out.

However, none of the above documents discloses any efforts to combine restylability and improved washout from hair in one polymer though. Only simple stylability or restylability is in focus.

There is a desire for measures which either raise the level of both restylability and washout behavior or at least obtain a good balance between restylability and washout behavior.

### SUMMARY OF THE INVENTION

The aim of the invention is to provide a hair styling and restyling composition comprising a cosmetically acceptable pressure sensitive adhesive polymer with restyling performance and improved washout from hair. The aim of the invention is further to provide a pressure sensitive adhesive polymer that gives restylable (non brittle-film forming) hold to hair and easy washout during cleansing when formulated as a wax replacement polymer in hair wax formulations. The aim of the invention is further to provide a process for preparing said pressure sensitive adhesive polymer. The aim of the invention is further to use of said pressure sensitive adhesive polymer for improving the restylability and washout behavior of a hair styling and restyling composition.

The aim of the present invention is achieved by a hair styling and restyling composition comprising a pressure sensitive adhesive polymer with glass transition temperatures of lower than −20° C., wherein said pressure sensitive adhesive polymer comprises the following monomers A) to D) and crosslinker E) in polymerized form:

A) a C₂-C₁₂ alkyl (meth)acrylate,
B) a vinyl ester of C₂-C₁₀ monocarboxylic acid,
optionally C) an ethylenically unsaturated carboxylic acid having 3 to 10 carbon atoms or anhydride thereof,
D) a hydrophilic monomer selected from the group consisting of a polyethyleneglycol mono(meth)acrylate and C₁-C₄ alkoxy-polyethyleneglycol mono(meth)acrylate, preferably methoxy- or ethoxy-polyethyleneglycol mono(meth)acrylate with a M_{w} of 500-3000 g/mol, and
E) a cosmetically acceptable crosslinker, preferably a (poly)alkenyl ether,
wherein the cosmetically acceptable crosslinker E) is used in amount of from 0.4 to 0.9 wt %, based on the sum of A) to E).

In another aspect, the present invention also relates to the above-mentioned pressure sensitive adhesive polymer with glass transition temperatures of lower than 20° C.

In another aspect, the present invention also relates to a process for preparing said pressure sensitive adhesive polymer by emulsion polymerization.

In another aspect, the present invention also relates to the use of the above-mentioned pressure sensitive adhesive polymer for improving the restylability and washout behavior of a hair styling and restyling composition.

Subjects of the present invention are to be found in the independent claims of the present document. The dependent claims represent more specific embodiments of the present invention.

### DETAILED DESCRIPTION OF THE INVENTION

Generally, the present invention relates to a hair styling and restyling composition comprising a pressure sensitive adhesive polymer with glass transition temperatures of lower than −20° C.

Unless defined otherwise, all technical and scientific terms used herein have the same meaning as commonly understood by one of ordinary skill in the art to which this invention belongs.

Expressions “a”, “an”, “the”, when used to define a term, include both the plural and singular forms of the term.

The present invention relates to a hair styling and restyling composition comprising a pressure sensitive adhesive polymer with glass transition temperatures of lower than −20° C., wherein said pressure sensitive adhesive polymer comprises the following monomers A) to D) and crosslinker E) in polymerized form:

A) a C₂-C₁₂ alkyl (meth)acrylate,
B) a vinyl ester of C₂-C₁₀ monocarboxylic acid,
optionally C) an ethylenically unsaturated carboxylic acid having 3 to 10 carbon atoms or anhydride thereof,
D) a hydrophilic monomer selected from the group consisting of a polyethyleneglycol mono(meth)acrylate and C₁-C₄ alkoxy-polyethyleneglycol mono(meth)acrylate preferably methoxy- or ethoxy-polyethyleneglycol mono(meth)acrylate with a M_{w} of 500-3000 g/mol, and
E) a cosmetically acceptable crosslinker, preferably a (poly)alkenyl ether,
wherein the cosmetically acceptable crosslinker E) is used in amount of from 0.4 to 0.9 wt %, based on the sum of A) to E).

More particularly, the present invention relates to a hair styling and restyling composition comprising a pressure sensitive adhesive polymer with glass transition temperatures of lower than −20° C., which comprises the following monomers A) to D) and crosslinker E) in polymerized form:

A) from 60 to 95 wt % of a C₂-C₁₂ alkyl (meth)acrylate,
B) from 1 to 20 wt % of a vinyl ester of C₂-C₁₀ monocarboxylic acid,
C) from 0 to 6.5 wt % of an ethylenically unsaturated carboxylic acid having 3 to 10 carbon atoms or anhydride thereof,
D) from 0.1 to 25 wt % of a hydrophilic monomer selected from the group consisting of a polyethyleneglycol mono(meth)acrylate and C₁-C₄ alkoxy-polyethyleneglycol mono(meth)acrylate with a M_{w} of 500-3000 g/mol, and
E) from 0.4 to 0.9 wt % of a cosmetically acceptable crosslinker, preferably a (poly)alkenyl ether,
wherein the weight percents are all based on the sum of A) to E).

In one particular embodiment, as monomer A) a suitable C₂-C₁₂ alkyl (meth)acrylate is preferably selected from the group consisting of C₂-C₈ alkyl (meth)acrylate, for example, ethyl acrylate, n-butyl acrylate, 2-ethylhexyl acrylate, n-butyl methacrylate, t-butyl methacrylate, isobutyl acrylate, isooctyl acrylate, or mixtures thereof.

Among these monomers, C₄-C₈ alkyl (meth)acrylate, for example, 2-ethylhexyl acrylate, n-butyl methacrylate, t-butyl methacrylate, isobutyl acrylate, isooctyl acrylate, or mixtures thereof, especially n-butyl acrylate or 2-ethylhexyl acrylate is more preferred.

Monomer A) is used in amount of preferably from 65 to 90 wt %, more preferably from 70 to 85 wt %, based on the sum of A) to E).

In another particular embodiment, as monomer B) a suitable vinyl ester of C₂-C₁₀ monocarboxylic acid is preferably selected from the group consisting of vinyl ester of C₂-C₈ monocarboxylic acid, for example, vinyl acetate, vinyl propionate, vinyl butyrate, vinyl valerate, vinyl hexanoate, vinyl 2-ethylhexanoate, and mixtures thereof.

Among these monomers, vinyl ester of C₂-C₆ monocarboxylic acid, for example, vinyl acetate, vinyl propionate, vinyl butyrate, vinyl valerate, vinyl hexanoate, or mixtures thereof, especially vinyl acetate is more preferred.

Monomer B) is used in amount of preferably from 2 to 15 wt %, more preferably from 5 to 10 wt %, based on the sum of A) to E).

In another particular embodiment, as monomer C) a suitable ethylenically unsaturated carboxylic acid having 3 to 10 carbon atoms or anhydride thereof is preferably selected from the group consisting of a mono-ethylenically unsaturated mono- or di-carboxylic acid having 3 to 8 carbon atoms or anhydride thereof, for example, acrylic acid, methacrylic acid, maleic acid, fumaric acid, crotonic acid, itaconic acid, (meth)acrylic anhydride, maleic anhydride, and mixtures thereof, more preferably acrylic acid and/or methacrylic acid.

Monomer C) is used in amount of preferably from 0.2 to 6.0 wt %, more preferably from 0.4 to 3.5 wt %, based on the sum of A) to E).

In another particular embodiment, as hydrophilic monomer D) a suitable polyethyleneglycol mono(meth)acrylate or C₁-C₄ alkoxy-polyethyleneglycol mono(meth)acrylate has a M_{w} of preferably from 700 to 1800 g/mol, more preferably from 800 to 1200 g/mol and is used in amount of preferably from 1 to 25 wt %, more preferably from 5 to 22 wt %, most preferably from 10 to 20 wt %, based on the sum of A) to E). Among them, methoxy- or ethoxy-polyethyleneglycol mono(meth)acrylate is more preferred.

In another particular embodiment, as cosmetically acceptable crosslinker E) a suitable (poly)alkenyl ether is preferably an allylic ether of a polyol, more preferably an allylic ether of a tetraol, most preferably an allylic ether of a C₅-tetraol, for example pentaerythritol allyl ether (PETAE) and is used in amount of preferably from 0.5 to 0.8 wt %, more preferably from 0.6 to 0.7 wt %, based on the sum of A) to E).

Furthermore, according to the present invention, A) to E) and amounts thereof mentioned above can be used in any combination with each other.

The present invention also relates to a pressure sensitive adhesive polymer with glass transition temperatures of lower than −20° C., which comprises the following monomers A) to D) and crosslinker E) in polymerized form:

A) a C₂-C₁₂ alkyl (meth)acrylate,
B) a vinyl ester of C₂-C₁₀ monocarboxylic acid,
optionally C) an ethylenically unsaturated carboxylic acid having 3 to 10 carbon atoms or anhydride thereof,
D) a hydrophilic monomer selected from a group consisting of a polyethyleneglycol mono(meth)acrylate and C₁-C₄ alkoxy-polyethyleneglycol mono(meth)acrylate, preferably methoxy- or ethoxy-polyethyleneglycol mono(meth)acrylate with a M_{w} of 500-3000 g/mol, and
E) a cosmetically acceptable crosslinker, preferably a (poly)alkenyl ether,
wherein the cosmetically acceptable crosslinker E) is used in amount of from 0.4 to 0.9 wt %, based on the sum of A) to E).

More particularly, the present invention also relates to a pressure sensitive adhesive polymer with glass transition temperatures of lower than −20° C., which comprises the following monomers A) to D) and crosslinker E) in polymerized form:

A) from 60 to 95 wt % of a C₂-C₁₂ alkyl (meth)acrylate,
B) from 1 to 20 wt % of a vinyl ester of C₂-C₁₀ monocarboxylic acid,
C) from 0 to 6.5 wt % of an ethylenically unsaturated carboxylic acid having 3 to 10 carbon atoms or anhydride thereof,
D) from 0.1 to 25 wt % of a hydrophilic monomer selected from a group consisting of a polyethyleneglycol mono(meth)acrylate and C₁-C₄ alkoxy-polyethyleneglycol mono(meth)acrylate with a M_{w} of 500-3000 g/mol, and
E) from 0.4 to 0.9 wt % of a cosmetically acceptable crosslinker, preferably a (poly)alkenyl ether,
wherein the weight percents are all based on the sum of A) to E).

A) to E) can be polymerized preferably by free-radical means or, where possible, anionically. As customary polymerization methods, both free-radical and anionic polymerization are familiar to the skilled worker.

Free-radical polymerization can be carried out, for example, in solution, such as in an organic solvent (solution polymerization), in aqueous dispersion (emulsion polymerization, suspension polymerization) or in bulk, i.e. essentially in the absence of water or organic solvents (bulk polymerization).

The pressure sensitive adhesive polymer is preferably prepared by emulsion polymerization.

The emulsion polymerization is carried out using ionic and/or nonionic emulsifiers and/or protective colloids and/or stabilizers as surface-active compounds.

Examples of suitable protective colloids are polyvinyl alcohols, starch derivatives and cellulose derivatives, or vinyl pyrrolidone copolymers. A detailed description of further suitable protective colloids is given in Houben-Weyl, Methoden der Organischen Chemie, Volume XIV/1, Makromolekulare Stoffe [macromolecular substances], Georg-Thieme-Verlag, Stuttgart 1961, pp. 411-420. Mixtures of emulsifiers and/or protective colloids may also be used. It is preferred as surface-active compounds to use exclusively emulsifiers, whose relative molecular weights are usually below 2000 g/mol. Preference is given to the use of at least one anionic emulsifier, alone or in combination with a nonionic emulsifier.

Anionic emulsifiers include alkali metal salts or ammonium salts of alkyl sulfates (alkyl: C₈-C₁₂), of dialkyl esters of sulfosuccinic acid (alkyl: C₄-C₁₀), of sulfuric monoesters with ethoxylated alkanols (EO units: 2 to 50, alkyl: C₁₂ to C₁₈) and with ethoxylated alkylphenols (EO units: 3 to 50, alkyl: C₄-C₁₀), of alkylsulfonic acids (alkyl: C₁₂-C₁₈) and of alkylarylsulfonic acids (alkyl: C₉ to C₁₈). The anionic surface-active compounds also include monoalkyl and dialkyl derivatives of sulfonylphenoxybenzenesulfonic acid salts, especially their sodium, potassium or calcium salts. The alkyl groups in these compounds have generally 6 to 18 and especially 6, 12 or 16 carbon atoms. Use is frequently made of technical mixtures containing a fraction of from 50 to 90 wt % of the monoalkylated product. These compounds are general knowledge, from U.S. Pat. No. 4,269,749, for example, and are obtainable commercially, for example, as Dowfax® 2A1 (trademark of the Dow Chemical Company).

Suitable nonionic emulsifiers are araliphatic or aliphatic nonionic emulsifiers, examples being ethoxylated mono-, di- and trialkylphenols (EO units: 3 to 50, alkyl: C₄-C₉), ethoxylates of long-chain alcohols (EO units: 3 to 50, alkyl: C₈-C₃₆), and also polyethylene oxide/polypropylene oxide block copolymers. Preference is given to ethoxylates of long-chain alkanols (alkyl: C₁₀-C₂₂, average degree of ethoxylation: from 3 to 50) and, of these, particular preference to those based on oxo alcohols and naturally occurring alcohols having a linear or branched C₁₂-C₁₈ alkyl radical and a degree of ethoxylation of from 8 to 50. Particularly, the nonionic emulsifiers include a polysorbate, and preferably polysorbate 20 or polysorbate 80 (commercially available as Tween 80).

Further suitable emulsifiers can be found in Houben-Weyl, Methoden der Organischen Chemie, Volume XIV/1, Makromolekulare Stoffe, Georg-Thieme-Verlag, Stuttgart, 1961, pp. 192-208.

The surface-active compounds preferably include alkali metal salts of C₈-C₁₂ alkyl sulfates or alkoxylated alkyl sulfates (EO units: 2 to 50, alkyl: C₁₂ to C₁₈), more preferably sodium lauryl sulfate, sodium lauryl ether sulfate, or polysorbate, and preferably polysorbate 20 or polysorbate 80 (commercially available as Tween 80), or mixtures thereof.

The surface-active compound is normally used in an amount of preferably from 0.1 to 10 wt %, more preferably from 0.5 to 5 wt %, based on weight of A) to E) that are to be polymerized. It is also possible to use a plurality of different surface-active compounds in the course of emulsion polymerization.

Examples of water-soluble initiators which can be used for emulsion polymerization are ammonium salts and alkali metal salts of persulfuric acid, e.g. sodium persulfate, hydrogen peroxide or organic peroxides, e.g. tert-butyl hydroperoxide.

Reduction-oxidation (redox) initiator systems are particularly suitable, consisting of at least one, usually inorganic reducing agent and of an inorganic or organic oxidizing agent.

The oxidation component comprises, for example, the above-mentioned initiators for emulsion polymerization.

The reduction component comprises, for example, alkali metal salts of sulfurous acid, such as sodium sulfite, sodium hydrogen sulfite, alkali metal salts of disulfurous acid, such as sodium disulfite, bisulfite addition compounds with aliphatic aldehydes and ketones, such as acetone bisulfite, or reducing agents such as hydroxymethanesulfinic acid and its salts, or ascorbic acid. The redox initiator systems can be used along with soluble metal compounds whose metallic component is able to exist in a plurality of valency states.

Examples of common redox initiator systems are ascorbic acid/iron (II) sulfate/sodium peroxodisulfate, tert-butyl hydroperoxide/sodium disulfite, and tert-butyl hydroperoxide/Na hydroxymethanesulfinate. The individual components, for example the reduction component, may also be mixtures, for example a mixture of the sodium salt of hydroxymethanesulfinic acid with sodium disulfite.

The above mentioned initiators are mostly employed in the form of aqueous solutions, the lower concentration being determined by the amount of water which is acceptable in the dispersion and the upper concentration by the solubility of the relevant compound in water. In general the concentration is from 0.1 to 30 wt %, preferably from 0.5 to 20 wt %, particularly preferably from 1.0 to 10 wt %, based on the solution.

The amount of initiators is generally from 0.01 to 10 wt %, preferably from 0.05 to 5 wt %, more preferably from 0.1 to 2 wt %, based on weight of A) to E) that are to be polymerized. It is also possible to use a plurality of different initiators in the course of emulsion polymerization.

In addition, regulators can also be added during polymerization, thereby reducing the molar mass. Examples of suitable regulators are compounds containing a thiol group, such as tert-butyl mercaptan, ethylacryl thioglycolate, mercaptoethanol, mercaptopropyltrimethoxysilane or tert-dodecyl mercaptan. The proportion of these regulators can be in particular from 0 to 0.3 wt %, preferably from 0.02 to 0.3 wt %, based on the polymer.

The emulsion polymerization is generally conducted at from 30 to 100° C., preferably from 50 to 95° C. The polymerization medium can consist either just of water or of mixtures of water and water-miscible liquids such as methanol. Preferably, only water is used. The emulsion polymerization can be carried out either batch wise or in the form of a feed process, including a stepwise or gradient procedure. Preference is given to the feed process, in which some of the polymerization batch is introduced as an initial charge, heated to the polymerization temperature and then initially polymerized, and then the remainder of the polymerization batch is supplied, in the course of continuing polymerization, to the polymerization zone continuously, stepwise or under a concentration gradient and usually by way of a plurality of spatially separate feed streams, of which one or more contain the monomers in pure or emulsified form.

The emulsion polymerization produces aqueous polymer dispersions with, in general, a solid content of from 15 to 75 wt %, preferably from 25 to 75 wt %.

The hair styling and restyling composition comprises from 0.1 to 20 wt %, preferably from 5 to 18 wt %, more preferably from 10 to 16 wt % the pressure sensitive adhesive polymer, based on weight of said hair styling and restyling composition.

The compositions of the present invention can also comprise any additive usually used in the field under consideration. For example, dispersants, antioxidants, essential oils, preserving agents, fragrances, liposoluble polymers that are dispersible in the medium, fillers, neutralizing agents, cosmetic and dermatological active agents such as, for example, emollients, moisturizers, vitamins, essential fatty acids, sunscreens, or mixtures thereof can be added. A non-exhaustive listing of such ingredients can be found in U.S. patent application publication no. 2004/0170586, the entire contents of which is hereby incorporated by reference. Further examples of suitable additional components can be found in the other references which have been incorporated by reference in this application. Still further examples of such additional ingredients may be found in the International Cosmetic Ingredient Dictionary and Handbook (9th ed. 2002).

A person skilled in the art will take care to select the optional additional additives and/or the amount thereof such that the advantageous properties of the composition according to the invention are not, or are not substantially, adversely affected by the envisaged addition.

These substances may be selected variously by the person skilled in the art in order to prepare a composition which has the desired properties.

These additives may be present in the composition in a proportion from 0 to 99% (such as from 0.01 to 90%) relative to the total weight of the composition and further such as from 0.1 to 50% (if present).

Needless to say, the composition of the invention should be cosmetically or dermatologically acceptable, i.e., it should contain a non-toxic physiologically acceptable medium and should be able to be applied to the hair of human beings. For the purposes of the invention, the expression “cosmetically acceptable” means a composition of pleasant appearance, odor, feel and/or taste.

The pressure sensitive adhesive polymer of present invention is used for improving the restylability and washout behavior of a hair styling and restyling composition.

The examples given below are intended to illustrate the invention without restricting it.

### EXAMPLES

### Polymer Synthesis

Exemplary Synthesis Protocol with Polymer P.2:

Preparation of pre-emulsion: To a stirred solution of surfactants (10 g, sodium lauryl sulfate and Tween 80) in de-mineralized water (130 mL), vinyl acetate (20.4 g), acrylic acid (1.3 g), 2-ethyl hexyl acrylate (205.8 g), methoxy-polyethyleneglycol monomethacrylate (Mw˜1000 g/mol, 25.4 g) and pentaerythrythyl allyl ether (1.5 g) is added and dispersed under stirring for 30 minutes.

A 2 L reactor fitted with condenser and stirrer is charged with demineralized water (373 mL), sodium lauryl sulfate (1.6 g) and heated to 85° C. under nitrogen atmosphere. At 85° C. sodium persulfate (0.4 g) in de-mineralized water (60 g) is added in one portion. The pre-emulsion is added slowly, so is a mixture of de-mineralized water (40 g) and sodium persulfate (0.3 g). After maintaining the temperature at 85° C. for a further period of 45 minutes, another portion of sodium persulfate (0.10 g) in de-mineralized water (15 mL) is added. The reaction temperature is maintained at 85° C. for another 180 minutes, then cooled to 50° C. and the product is filtered over steel gauze filter (125 mesh) to get the product as an off-white emulsion with a solid content of about 30%.

### Chassis Formulation (all Amounts are Active Concentrations)

15 wt % of wax or polymer were formulated according to the following recipe to determine their performance regarding restylability and washout behavior.

| Amount [wt %] | Ingredient | INCI | Supplier |
|---|---|---|---|
| 15 | Polymer/Wax | | |
| 8 | Xiameter PMX-245 | Cyclomethicone | Dow Corning |
| 8 | Luvitol EHO | Cetearyl Ethylhexanoate | BASF |
| 3 | Vaseline | Petrolatum | SCR |
| 3 | Edenor C1898 | Stearic Acid | Longwen |
| | | | Chemicals |
| 3 | Eumulgin SMS 20 | Polysorbate 60 | BASF |
| 0.25 | Luvigel Fit | Acrylates C₁₀-C₃₀ Alkyl | BASF |
| | | Methacrylates Copolymer | |
| 1 | Cutina GMS | Glyceryl Stearate | BASF |
| q.s. | Triethanolamine Care | Triethanolamine | BASF |
| 0.05 | EDETA BD | Disodium EDTA | BASF |
| Add 100 | Water | Aqua | |

### Measurement of Restylability

Standard 2 g hair tresses (Asian origin) were washed and dried over night at 23° C. and 50% relative humidity. 4 tresses per product were used for the restyling test. 500 mg of product formulation were applied on the top of each hair tress and distributed evenly. The tress were flattened by pulling it between the thumb and the slightly bend index finger and hanged to dry for 4 h at 23° C. and 50% relative humidity. Then the initial hold was measured by a regular 3-point bending stiffness test. After this test, the hair tresses were completely bend several times to mimic a restyling, flattened again as before by pulling it between the thumb and the slightly bend index finger and the residual hold at 4 h was determined by another 3-point bending stiffness test. The same procedure was done after 24 h.

### Measurement of Washout Behavior (Sensory Assessment)

Standard 2 g hair tresses (single bleached, Asian origin) are treated with 600 mg of the hair wax formulations and dried for 4 hours. Then the hair tresses are washed under identical conditions and tresses treated with different formulations are compared by a sensory assessment panel on their remaining residues and cleanness.

### Restyling Results

As shown in the table below, polymers P.2-P.6 and C.1-C.3 showed a higher initial hold and similar or better restyled hold after 4 h and 24 h compared to Candelilla wax containing formulations W.1, wherein polymer P.6 was synthesized from large scale available MPEG methacrylate that contains minor amounts of methacrylic acid as a side product. Polymer P.1 has a worse restyled hold after 4 h and 24 h. As mentioned before, all market products which are film forming polymers, e.g. PVP Luviskol K30 (BASF) and also the products that claim to have restylability (Plascize products, Goo Chemicals) did not show any observable hold after restyling at 4 h or 24 h in this measurement.

| | | | | | | | Viscosity of | Initial | 4 h | 24 h |
|---|---|---|---|---|---|---|---|---|---|---|
| | Composition [wt %] | | | | | | formulation | Hold | restyling | restyling |
| Number | EHA | VA | AA | MAA | PETAE | MPEG-MA | [Pas] | [cN] | [cN] | [cN] |
| P.1 | 81.2 | 8 | 0.5 | — | 0.3 | 10 | 88 | 84 | 33 | 32 |
| P.2 | 80.9 | 8 | 0.5 | — | 0.6 | 10 | 67 | 79 | 40 | 37 |
| P.3 | 80.7 | 8 | 0.5 | — | 0.8 | 10 | 71 | 106 | 45 | 39 |
| P.4 | 80.5 | 8 | 0.5 | — | 1 | 10 | 77 | 119 | 49 | 44 |
| P.5 | 70.9 | 8 | 0.5 | — | 0.6 | 20 | 112 | 94 | 46 | 43 |
| P.6 | 70.9 | 8 | 0.5 | 2.8 | 0.6 | 17.2* | 100 | 83 | 43 | 39 |
| C.1 | 90.9 | 8 | 0.5 | — | 0.6 | — | 69 | 85 | 40 | 39 |
| C.2 | 90.7 | 8 | 0.5 | — | 0.8 | — | 40 | 96 | 40 | 41 |
| C.3 | 90.5 | 8 | 0.5 | — | 1 | — | 81 | 114 | 46 | 45 |

| | | | | | |
|---|---|---|---|---|---|
| W.1 | Candelilla Wax | 107 | 74 | 38 | 41 |
| W.3 | Microcrystalline Wax | 78 | 68 | 41 | 41 |
| W.4 | Beewax | 108 | 63 | 40 | 40 |
| M.1 | Luviskol K30 (PVP, BASF) | 36 | 178 | <20 | <20 |
| M.2 | Plascize L2714 (Goo Chemicals) | 130 | 31 | <20 | <20 |
| M.3 | Plascize L2700 (Goo Chemicals) | 94 | 55 | <20 | <20 |

EHA: 2-Ethylhexyl acrylate,
VA: Vinyl acetate,
AA: Acrylic acid,
MAA: Methacrylic acid,
PETAE: Pentaerythrytyl allylether,
MPEG-MA: Methoxy-polyethyleneglycol monomethacrylate, M_{w} ~1000 g/mol;
Viscosity data: Brookfield RV7, 10 rpm, 23° C.;
*means a value with the exclusion of MAA.

### Washout Results

Candelilla wax containing formulation W.1 served as benchmark to compare the washout behavior. Comparative examples C.2 and C.3 showed a worse washout than W.1. Introduction of a hydrophilic monomer (MPEG-MA) and lowering the amount of crosslinker to 0.8 (P.3) and 0.6 (P.2) led to polymers with high restyling performance and similar washout in comparison with wax formulation W.1. Higher amounts of crosslinker (P.4) lead to worse washout. Increasing the amount of hydrophilic monomer to 20 wt % and keeping the crosslinker at 0.6 wt % (P.5) gave the best results and showed a much better washout than Candelilla wax containing formulations. Also polymer P.6 showed much better washout than Candelilla wax containing formulations.

| | Composition [wt %] | | | | | | Washout behavior compared |
|---|---|---|---|---|---|---|---|
| Number | EHA | VA | AA | MAA | PETAE | MPEG-MA | to Candelilla Wax W.1 |
| P.1 | 81.2 | 8 | 0.5 | — | 0.3 | 10 | n.d. |
| P.2 | 80.9 | 8 | 0.5 | — | 0.6 | 10 | better |
| P.3 | 80.7 | 8 | 0.5 | — | 0.8 | 10 | similar |
| P.4 | 80.5 | 8 | 0.5 | — | 1 | 10 | worse |
| P.5 | 70.9 | 8 | 0.5 | — | 0.6 | 20 | much better |
| P.6 | 70.9 | 8 | 0.5 | 2.8 | 0.6 | 17.2* | much better |
| C.2 | 90.7 | 8 | 0.5 | — | 0.8 | — | worse |
| C.3 | 90.5 | 8 | 0.5 | — | 1 | — | worse |

| | | |
|---|---|---|
| W.1 | CandelillaWax | benchmark |
| W.3 | Microcrystalline Wax | n.d. |
| W.4 | Beewax | n.d. |
| M.1 | Luviskol K30 (PVP) | n.d. |
| M.2 | Plascize L2700/L2714 | n.d. |

Abbreviations mentioned above apply.
n.d. not determined.
*means a value with the exclusion of MAA.

Except in the Examples, or where otherwise explicitly indicated, all numerical quantities in this description specifying amounts of materials, reaction conditions, molecular weights, number of carbon atoms, and the like, are to be understood as modified by the word “about”.

It is to be understood that the upper and lower amount, range, and ratio limits set forth herein may be independently combined. Similarly, the ranges and amounts for each element of the invention can be used together with ranges or amounts for any of the other elements.

The present invention is not to be limited in scope by the specific embodiments and examples described herein. Indeed, various modifications of the invention in addition to those described herein will become apparent to those skilled in the art from the foregoing description. Such modifications are intended to fall within the scope of the appended claims.

### Further Examples

In order to compare the polymers according to the present invention with the polymer disclosed in example 7 of EP 2 611 412 A2 the following the following experiments were carried out. The polymer according to example 7 of EP 2 611 412 A2 was synthesized. Its physical parameters and performance was analyzed. The following was found:
1) The glass transition temperature of 76° C. is far above the glass transition temperature of the polymers according to the present invention.
2) Due to the high amount of methacrylic acid (34.5 wt-%) in combination with the crosslinker, the polymer acts as a strong thickener upon neutralization. Due to this strong thickening effect it was not possible to formulate a hair wax formulation with an acceptable viscosity. Furthermore the obtained texture made it impossible to properly distribute the formulation on hair and there was no hold detectable.
3) In summary, the polymer according to example 7 of EP 2 611 412 A2 is not suitable for the application for which the polymer according to the present invention can be used.

| | Glass Transition | Viscosity | | Viscosity | |
|---|---|---|---|---|---|
| | Temperature | (10% active, pH 6.6) | | (15% active, pH 6.6) | |
| | Tg [° C.] | [mPas] | | [mPas] | |
| P.6 | <−40 | 57 | (RV2, 100 rpm) | 61 | (RV2, 100 rpm) |
| (according to the | | | | | |
| present invention) | | | | | |
| “Polymer 7” | 76 | 230 000 | (R7, 10 rpm) | 2 100 000 | (RV7, 1 rpm) |
| (according to | | | | | |
| example 7 of EP | | | | | |
| 2 611 412 A2) | | | | | |

1. A pressure sensitive adhesive polymer with a glass transition temperature of lower than −20° C., wherein said pressure sensitive adhesive polymer comprises monomers A) to D) and crosslinker E) in polymerized form:
A) a C₂-C₁₂ alkyl (meth)acrylate,
B) a vinyl ester of C₂-C₁₀ monocarboxylic acid,
optionally C) an ethylenically unsaturated carboxylic acid having 3 to 10 carbon atoms or anhydride thereof,
D) a hydrophilic monomer selected from the group consisting of a polyethyleneglycol mono(meth)acrylate and C₁-C₄ alkoxy-polyethyleneglycol mono(meth)acrylate with a M_{w} of 500-3000 g/mol, and
E) a cosmetically acceptable crosslinker, preferably a (poly)alkenyl ether,
wherein the cosmetically acceptable crosslinker E) is used in amount of from 0.4 to 0.9 wt %, based on the sum of A) to E).

2. The polymer according to claim 1, comprising
A) from 60 to 95 wt %, of a C₂-C₁₂ alkyl (meth)acrylate,
B) from 1 to 20 wt %, of a vinyl ester of C₂-C₁₀ monocarboxylic acid,
C) from 0 to 6.5 wt %, of an ethylenically unsaturated carboxylic acid having 3 to 10 carbon atoms or anhydride thereof,
D) from 0.1 to 25 wt %, of the hydrophilic monomer according to claim 1, and
E) from 0.4 to 0.9 wt %, a cosmetically acceptable crosslinker,
wherein the weight percents are all based on the sum of A) to E).

3. The polymer according to claim 1, wherein the monomer A) is a C₂-C₈ alkyl (meth)acrylate.

4. The polymer according to claim 1, wherein the monomer B) is a vinyl ester of C₂-C₈ monocarboxylic acid.

5. The polymer according to claim 1, wherein the monomer C) is selected from the group consisting of mono- or di-ethylenically unsaturated mono-carboxylic acids having 3 to 8 carbon atoms or anhydrides thereof.

6. The polymer according to claim 1, wherein the monomer D) is a C₁-C₄ alkoxy-polyethyleneglycol mono(meth)acrylate having a M_{w} of from 700 to 1800 g/mol.

7. The polymer according to claim 1, wherein the crosslinker E) is an allylic ether of a polyol.

8. A process for preparing the polymer according to claim 1 by emulsion polymerization.

9. The process according to claim 8, wherein the emulsion polymerization is carried out using ionic and/or nonionic emulsifiers and/or
protective colloids and/or stabilizers as surface-active compounds.

10. The process according to claim 9, wherein the surface-active compounds comprise alkali metal salts of C₈-C₁₂ alkyl sulfates, C₈-C₁₂ alkyl ether sulfates, sodium lauryl ether sulfates, or polysorbate, or mixtures thereof.

11. The process according to claim 8, wherein the emulsion polymerization is carried out using water-soluble initiators.

12. A method for improving a restylability and washout behavior of a hair styling and restyling composition comprising the use of a polymer according to claim 1.

13. A hair styling and restyling composition comprising a polymer according to claim 1.

14. The hair styling and restyling composition according to claim 13, comprising from 0.1 to 20 wt %, of the polymer, based on the weight of said hair styling and restyling composition.

15. The polymer according to claim 1, comprising
A) from 65 to 90 wt %, of a C₂-C₁₂ alkyl (meth)acrylate,
B) from 2 to 15 wt %, of a vinyl ester of C₂-C₁₀ monocarboxylic acid,
C) from 0.2 to 6.0 wt %, of an ethylenically unsaturated carboxylic acid having 3 to 10 carbon atoms or anhydride thereof,
D) from 1 to 25 wt %, of the hydrophilic monomer according to claim 1, and
E) from 0.5 to 0.8 wt %, of a cosmetically acceptable crosslinker,
wherein the weight percents are all based on the sum of A) to E).

16. The polymer according to claim 3, wherein the monomer A) is selected ethyl acrylate, n-butyl acrylate, 2-ethylhexyl acrylate, n-butyl methacrylate, t-butyl methacrylate, isobutyl acrylate, isooctyl acrylate, or mixtures thereof, preferably C4-C8 alkyl (meth)acrylates, for example, 2-ethylhexyl acrylate, n-butyl methacrylate, t-butyl methacrylate, isobutyl acrylate, isooctyl acrylate, or mixtures thereof, especially n-butyl acrylate, and 2-ethylhexyl acrylate.

17. The polymer according to claim 4, wherein the monomer B) is selected from the group consisting of vinyl acetate, vinyl propionate, vinyl butyrate, vinyl valerate, vinyl hexanoate, vinyl 2-ethylhexanoate, or mixtures thereof, preferably vinyl esters of C₂-C₆ monocarboxylic acid, for example, vinyl acetate, vinyl propionate, vinyl butyrate, vinyl valerate, vinyl hexanoate, and mixtures thereof.

18. The polymer according to claim 4, wherein the monomer C) is selected from acrylic acid, methacrylic acid, maleic acid, fumaric acid, crotonic acid, itaconic acid, (meth)acrylic anhydride, maleic anhydride, or mixtures thereof.

19. The polymer according to claim 7, wherein the crosslinker E) is an allylic ether of a tetraol.

20. The process according to claim 11, wherein the water-soluble initiator comprises an ammonium salt or an alkali metal salt of persulfuric acid, hydrogen peroxide, or an organic peroxide.
